(19) United States
(12) Patent Application Publication
(10) Pub. No.: US 2014/0127323 A1
(43) Pub. Date: May 8, 2014

(54) DIETETIC MULTI-COMPONENT SYSTEM

(71) Applicant: Martin HULLIGER, Zuerich (CH)

(72) Inventor: Martin HULLIGER, Zuerich (CH)

(21) Appl. No.: 14/153,528

(22) Filed: Jan. 13, 2014

Related U.S. Application Data
(63) Continuation of application No. PCT/CH2012/000166, filed on Jul. 13, 2012.

(30) Foreign Application Priority Data
Jul. 13, 2011 (EP) 11005726.2

(51) Int. Cl. A61K 9/00; A23L 1/29

(52) U.S. Cl. CPC A61K 9/0053; A23L 1/296
USPC 424/637

(57) ABSTRACT

An improved multi-component system containing a main component and at least one additional component that is physically separated from the main component, and a use of the system as a food supplement and a pharmaceutical product.

This nonprovisional application is a continuation of International Application No. PCT/CH2012/000166, which was filed on Jul. 13, 2012, and which claims priority to European Patent Application No. 11005726.2, which was filed on Jul. 13, 2011, and which are both herein incorporated by reference.

### BACKGROUND OF THE INVENTION

1. Field of the Invention

The present invention relates to an improved multi-component system, containing a main component and at least one additional component physically separated therefrom, and the use thereof as a food supplement and pharmaceutical product. The invention relates further to the use staggered over time (“dosing schedule”) of said multi-component system and the extracellular action of said multi-component system.

2. Description of the Background Art

It is known that the human body requires a great number of different nutrients, including a minimum of 60 minerals, 15 vitamins, 12 essential amino acids, and 3 essential fatty acids.

It is known further that the uptake of minerals is pH-dependent. The following order, for instance, can be provided for a suitable pH range: Na, Mg>Ca, K>Mg, Fe>Zn, Cu>I; this means that sodium can be taken up by the body over a very broad pH range, whereas the iodine uptake occurs only in a very narrow pH range.

It is known further that the uptake of other nutrients, such as vitamins, amino acids, and fatty acids, is subject to a complex interaction and because of biological processes is active to a different extent at different times of day (“biorhythm”).

One-component systems and multi-component systems, adapted to human needs, as food supplements or pharmaceutical products are known and widespread.

Saltman et al. (U.S. Pat. No. 5,151,274) describes a formulation, also in the form of a multi-component system, comprising calcium and trace elements, for improving bone growth and treating age-related bone loss.

Collins (U.S. 2003/0018009) describes a formulation with an increased vitamin B12 content and the use thereof for the treatment of autoimmune diseases.

Rüsing et al. (WO2004/105516), which corresponds to US 20070010480, describes a dietetic formulation containing at least one n3 fatty acid and at least one dietary fiber and the use thereof for weight control or weight reduction.

Gervais et al. (CA2478278) describes a supplement with at least two different dosage units containing vitamins, calcium carbonate, and other components.

Riley (U.S. Pat. No. 5,976,658) describes a modular system of food supplements for promoting health, likewise with at least two different dosage units containing vitamins, calcium carbonate, and other components.

The known formulations are regarded as insufficient or disadvantageous for various reasons. Thus, these are often targeted to very specific actions and/or are limited in their use and/or management.

### SUMMARY OF THE INVENTION

It is therefore an object of the present invention to reduce the disadvantages of the prior-art formulations. In particular, it is an object of the present invention to provide formulations that have a broad field of application, are easy to use, and/or can be managed flexibly.

It is of particular importance to improve the uptake of valuable nutrients in order to thus have a positive effect on the human body. It is advantageous further to provide a flexibly manageable system in order to thus satisfy special requirements. A further object of the present invention is to improve existing treatment options.

The heretofore outlined objects are attained according to the independent claims. The dependent claims describe advantageous embodiments.

As long as no other meaning arises from the immediate context, the following terms may have the meaning stated herein.

The general, preferred, and especially preferred embodiments, ranges, etc., given in connection with the present invention, can be combined as desired with one another. Likewise, individual definitions, embodiments, etc., can be omitted or not be relevant.

The term “cosmetic treatment” is to be construed to mean that it does not comprise a “therapeutic treatment.” Accordingly, the terms “cosmetic formulation” are to be differentiated from a “therapeutic formulation.” The term “treatment” comprises the use of the formulation described herein as monotherapy, as combination therapy, and supportive/concomitant treatment (“co-therapy”).

Percentages are given as % by weight, unless noted otherwise.

The term “multi-component system” is known and comprises dosage forms that comprise two or more individual (i.e., physically separated) compositions. Such multi-component systems are especially suitable for taking the individual components independently of one another, e.g., according to a predetermined schedule, as described hereafter.

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The present invention will become more fully understood from the detailed description given hereinbelow and the accompanying drawings which are given by way of illustration only, and thus, are not limitive of the present invention, and wherein:

FIG. 1 shows in a schematic manner different embodiments of the multi-component system of the invention; GK denotes the main component and ZK the additional component(s);

FIGS. 2a-c show in schematic manner different dosing schedules for a main component (GK) and one or more additional component(s) (ZK);

FIGS. 3a-d show in schematic manner different dosing schedules for a GK and a plurality of ZK; and

FIGS. 4a-d show in schematic manner different dosing schedules for a GK and a plurality of ZK.

### DETAILED DESCRIPTION

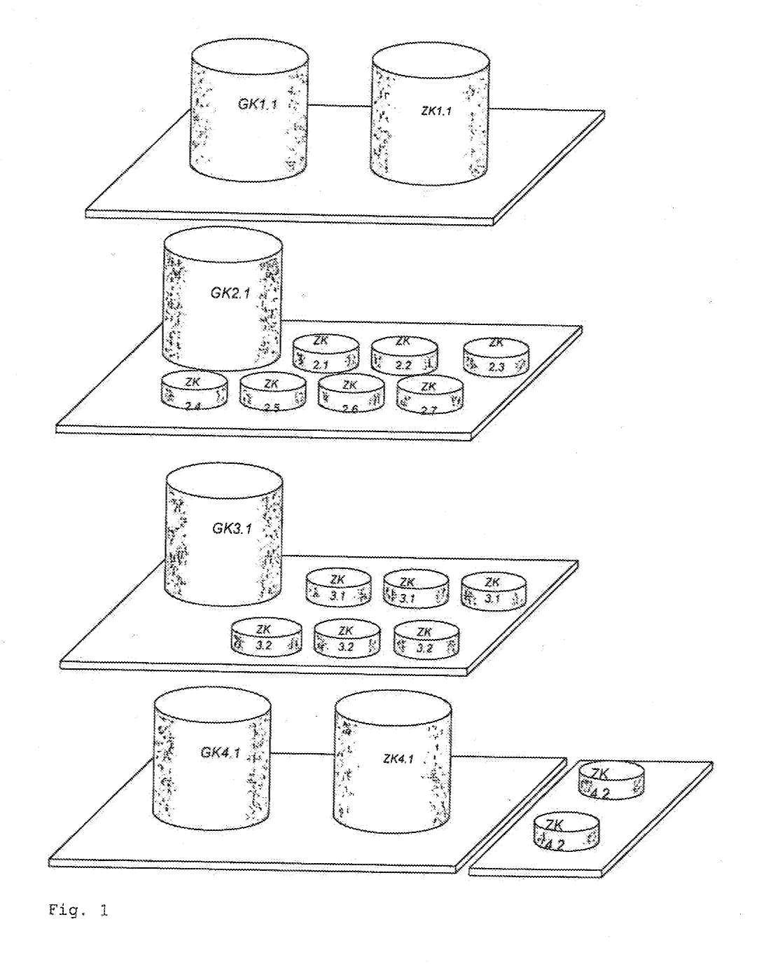

FIG. 1, at the top, shows, in a simplest embodiment of the multi-component system described herein, a formulation for the main and additional component in physically separated formulations.

FIG. 1, 2^{nd} row, shows in an alternative embodiment of the multi-component system described herein a formulation for the main and additional component in physically separated formulations. In this embodiment, the GK is dosed individually (for example, as a powder), and the ZK is given in ready-to-use pre-dosed formulations (for example, as an effervescent tablet).

FIG. 1, 3^{rd} row, shows in an alternative embodiment of the multi-component system described herein a formulation for a main component and two additional components in physically separated formulations. In this embodiment, the GK is dosed individually (for example, as a powder), and the additional components are given in ready-to-use pre-dosed formulations (for example, as a syrup).

FIG. 1, at the bottom, shows in an alternative embodiment of the multi-component system described herein a formulation for a main component and an additional component in physically separated formulations analogous to the topmost row. In this embodiment, another ZK is dosed individually in addition (for example, as a powder); this additional component need not absolutely correspond to the additional components described herein; it can also be a gas (for example, oxygen) or a pharmaceutical preparation.

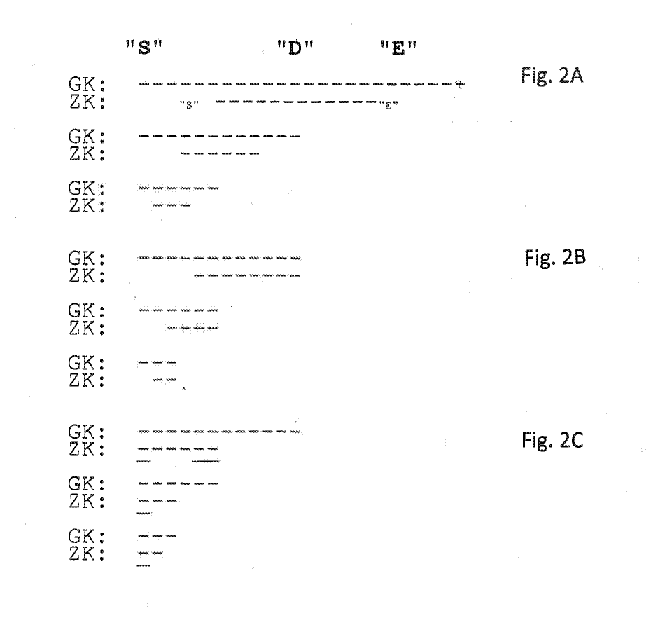

FIG. 2 shows in schematic manner different dosing schedules for a main component (GK) and one or more additional component(s) (ZK). An en dash symbolizes the time period of % month. Within the dosing interval, the dosage of GK and ZK can remain constant or vary. The details are variable preferably from each of the indicated points (“S” “D” “E”) up to 30% (“S” “D” “E”)+/−, 50% (“S” “D” “E”)+/−, 70% (“S” “D” “E”)+/−, from “S” Start/“D” duration/“E” end of the GK+ZK(1).

In FIG. 2 A, dosing schedules are shown in which the basic interval, therefore the dosing period for GK, is 12 or 6 or 3 months and the dosing period for the ZK is 50% of this time.

In FIG. 2 B, dosing schedules are shown in which the basic interval is 6 or 3 or 1.5 months and the dosing period for the ZK is ⅔ of this time.

In FIG. 2 C, dosing schedules are shown in which the basic interval is 6 or 3 or 1.5 months and the dosing period for the ZK is ⅓ of this time.

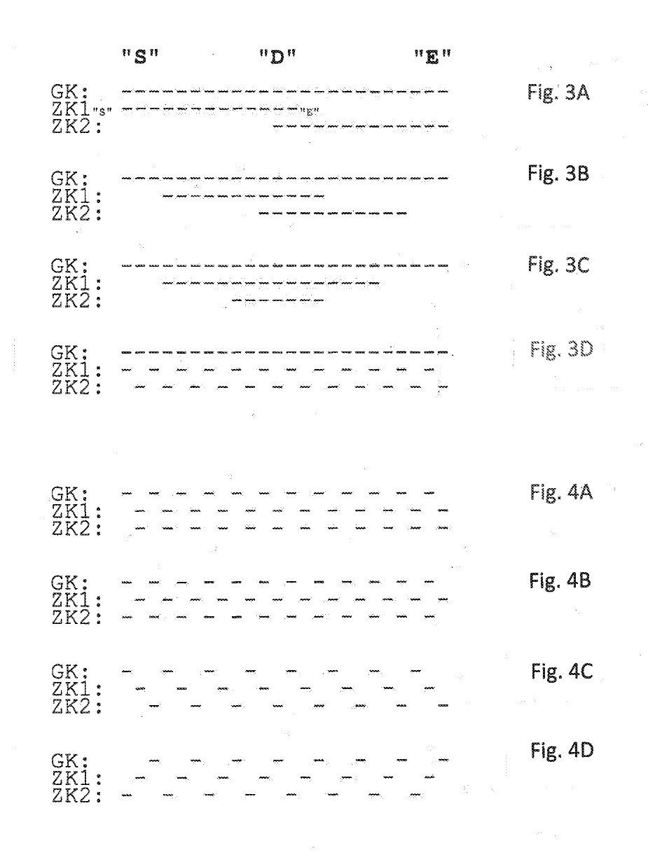

FIG. 3 shows in schematic manner different dosing schedules for a GK and a plurality of ZK. An en dash symbolizes the time period of ½ month. Within the dosing interval, the dosage of GK and ZK can remain constant or vary. According to these dosing schedules, two ZK are administered, whereby these ZK are administered sequentially (3A), administered partially overlapping (3B), administered totally overlapping (3C), or administered alternately (3D). In this case, the details for 5-95% for “S”+/−, “D”+/−, “E”+/−, of GK/ZK1 are variable.

FIG. 4 shows in schematic manner different dosing schedules for a GK and a plurality of ZK. An en dash symbolizes the time period of ½ month. Within the dosing interval, the dosage of GK and ZK can remain constant or vary. According to these dosing schedules, two ZK are administered; in this case, the details for 5-95% for “S”+/−“D”+/−, “E”+/−, of GK/ZK1 are variable.

The invention in a first aspect relates to a formulation/a product in the form of a multi-component system containing a main component (GK) and one or more additional components (ZK), physically separated therefrom, characterized in that the main component contains basic electrolytes and optionally silicates and the additional component contains vitamins and optionally trace elements, optionally amino acids/proteins, optionally plant extracts, and optionally other ingredients.

The formulation of the invention, particularly the individual components thereof, will be described in greater detail hereafter.

It was found surprisingly that the separate administration of nutrients, enabled by the separation into a main component and additional component(s), results in a number of advantages. Thus, it is possible, for example, with the formulation described herein/the product described herein
to achieve a much higher nutrient uptake than is possible with known products; and/or
to achieve a much better effect than is possible with known products; and/or
to provide a product adapted especially to the elimination of an undesirable condition/to the therapy of a disease; and/or
to achieve positive effects that are made possible only by the separate administration of nutrients; and/or
to increase the action of medications; and/or
to break down/excrete/neutralize medication-induced harmful products/by-products.

An element of the present invention is that the formulation exists in the form of a main component and one or more additional components, whereby said components are separated from one another. Such a system can also be described as a “Kit of Parts” comprising the main and additional component(s). The described division enables a sequential and independent administration of the main and additional component(s). This in turn enables an adaptation of the system to the desired goal (as described hereafter) and the administration of ingredients incompatible per se. The terms main and additional component are not to be taken to mean that one of the two is quantitatively greater than the other, nor that commercial products are designated as the “main component” and “additional component.” Rather, these terms are to be understood neutrally as “a first component” and “one or more other components.”

The term “basic electrolytes” is known and comprises alkali salts and alkaline earth salts, such as carbonates, hydrogen carbonates, sulfates, tartrates, hydrogen tartrates, both anhydrous and also as hydrates. These can be a solid and/or liquid (dissolved). In particular, these comprise sodium carbonate, sodium bicarbonate, calcium carbonate, calcium bicarbonate, magnesium carbonate, magnesium bicarbonate, magnesium sulfate, sodium/potassium tartrate, sodium tartrate, and potassium tartrate. Sodium carbonate and sodium bicarbonate are preferred; sodium bicarbonate is especially preferred. The GK contains the basic electrolytes described herein in an amount greater than 50% by weight, preferably greater than 66% by weight, especially preferably greater than 90% by weight. It has proven especially advantageous further if the GK contains at least 10% by weight, preferably at least 15% by weight, especially preferably at least 30% by weight of said sodium carbonate and/or sodium bicarbonate.

The term “silicates” is known and comprises synthetically produced silicates as well as silicates of natural origin, particularly from the group of diatomaceous earth.

The term “vitamins” is known and comprises in particular vitamin A, group B vitamins, vitamin C, vitamin D, vitamin E, and vitamin K.

The individual ingredients can be combined as desired with the vitamins.

The term “trace elements” is known and comprises in particular Cu-, Fe-, Zn-, Mn-, Cr-, Mo-, Se-, and I-containing compounds. Thus, “trace elements” differ from the aforementioned basic electrolytes, which are to be assigned to “major elements.”

The terms “amino acid” and “protein” are known and comprise proteinogenic amino acids, and other amino acids and proteins, suitable for human consumption or pharmaceutical use, and the metabolites thereof, particularly carnitine, creatinine, and taurine. Amino acids and proteins can be used as isolated substances and/or as natural products, rich in these amino acids/proteins. Amino acids/proteins can be present as an individual compound or as a mixture.

The term “plant extracts” is known and comprises extracts, suitable for human consumption or pharmaceutical use, of plants from essences obtained from leaves, flowers, shoots, roots, and/or fruit, particularly aqueous or alcoholic essences. Plant extracts selected from the group comprising guarana, caffeine, and maca are preferred. Plant extracts can be used as isolated substances (for example, caffeine) and/or as natural products, rich in these plant extracts (for example, green tea extract).

The term “other ingredients” comprises compounds that are suitable for human consumption or pharmaceutical use and cannot be subsumed under the aforementioned terms “vitamins,” “trace elements,” “amino acids/proteins,” or “plant extracts” and have a positive effect on the human body. The term comprises flavoring agents, aromatic substances, hormones, and hormone-like compounds, particularly hormones.

The formulation of the invention may but need not contain other food additives in its GK or ZK. “Food additives” are generally known based on their “E number” and comprise thereby particularly substances that positively influence the shelf life, color, taste, and processability.

As already mentioned, the formulation of the invention is suitable as a food supplement (“supplement”) and/or as a pharmaceutical product. It is understood that the formulation is adapted accordingly to these intended uses. This type of adaptation is within the scope of average knowledge and ability of a specialist. In particular, the main component and the additional components can be present independently of one another as a solid dosage form and as a liquid dosage form in the formulation of the invention. Further, the dosage forms can be adapted for oral or transdermal formulations.

Solid oral dosage forms comprise in particular powders, tablets, and capsules.

Liquid oral dosage forms comprise in particular solutions such as beverages, syrups, suspensions, and aerosols.

Transdermal dosage forms comprise in particular ointments, creams, and oils.

Furthermore, the formulations of the invention can be contained in gastro-resistant capsules, in homeopathic formulations, natural medicines, and/or emergency supplies of all types.

The main and additional component of the formulation of the invention can be taken either directly, for example, as a tablet or in the form of drops, or they can be incorporated into an end product, for example, as a powder or effervescent powder in a beverage, as a snack product, or into some other food.

The proportions by weight of the individual components in the GK and ZK can vary over broad ranges. In an advantageous embodiment, the invention relates to a formulation as described herein, characterized in that 10-100%, preferably 50-99% basic electrolyte is present in the main component and/or that 0.01-1500% of the daily dose that WHO recommends for this vitamin is present in the additional component, 30-500% being preferred and 50-300% being especially preferred.

In an embodiment, the content of a vitamin in the ZK is 15-60% of the daily dose recommended by WHO, provided said ZK is formulated as an effervescent tablet.

In a further embodiment, the content of a vitamin in the ZK is 5-70% of the daily dose recommended by WHO, provided said ZK is not formulated as an effervescent tablet.

In a further embodiment, a dosage of the main component is provided so that one of the basic electrolytes, preferably the major elements, is dosed in an amount greater than 300 mg per day.

In a further embodiment, the main component contains no, or substantially no, vitamins.

In a further embodiment, the additional component(s) contains (contain) no, or substantially no, basic electrolytes.

The individual components of GK and ZK are known and commercially available or can be prepared by known methods.

The main component and additional component(s) can be prepared from these individual components by known methods used in food technology and/or pharmaceutical technology, for example, by mixing, dispersing, or dissolving the individual components. In a further advantageous embodiment, the invention relates to a formulation prepared by the method described herein.

In an embodiment, the multi-component system described herein (“the product”) is a high-volume product in which major elements are provided to their full extent; this signifies a difference compared with the prior art.

In an embodiment, the product described herein is a high-volume product in which major elements are provided with an increased proportion of sodium carbonate/sodium bicarbonate, which signifies a difference compared with the prior art.

In an embodiment, the product described herein is a high-volume product in which major elements are provided according to a long-term plan within the range desired here for longer than a week/month, which signifies a difference compared with the prior art.

In an embodiment, the product described herein is a high-volume product in which major elements are provided according to a long-term plan in the range desired here for longer than a week; these allow the optimal low-harm flow of toxins through the body; this signifies a difference compared with the prior art.

The invention in a second aspect relates to the use the formulation of the invention as a food supplement and/or as a food. The uses described herein are based on the marked extracellular action of the formulation of the invention; said action becomes clear primarily when the dosing schedules described herein are followed.

The formulation of the invention can be used as a food supplement, particularly to support the achievement of diet goals and/or to promote the health of the human body in diverse ways. Said aspect of the invention will be described in greater detail hereafter.

The term “food supplement” is known and comprises such formulations that can be added to the human diet. Food supplements are suitable for an increased/improved supplying of the human body with the nutrients cited herein and/or for the supportive treatment to prevent negative conditions. Food supplements contain, in comparison with the aforementioned formulations, still further components which facilitate administration, processing, and/or preparation. Fillers, dyes, binders, and other components familiar to the person skilled in the art are mentioned.

The term “food” is known and comprises formulations that are used for human nutrition. The food can exist in liquid formulation, for example, as an alcohol-free beverage, as an alcohol-containing beverage, or in solid formulation, for example, as a snack product or cereals. Accordingly, in comparison with the aforementioned formulations, the food contains still further components such as water, flavorings, dyes, fats, proteins, carbohydrates, and optionally other components familiar to the person skilled in the art.

The invention relates further to a formulation as described herein to achieve a cosmetic (i.e., non-therapeutic) anti-aging effect or the use of a formulation as described herein as an anti-aging agent. It is especially advantageous for this use, if the additional component contains amino acids, particularly selected from the group comprising proline (e.g., from gelatin, mussel powder, whey protein, cheese, wheat germ, orange juice), glycine (e.g., from gelatin, mussel powder, whey protein, oats, beef), lysine (e.g., from gelatin, wheat germ, amaranth), arginine (e.g., from gelatin, wheat germ), methionine (e.g., from whey protein, oats), and cysteine (e.g., from oats, corn, egg whites, whey protein). In an advantageous embodiment, therefore, the present invention relates to a multi-component system as described herein in which the additional component contains amino acids selected from the group comprising proline, glycine, lysine, arginine, methionine, and cysteine.

The invention relates further to a formulation as described herein for the cosmetic (i.e., non-therapeutic) treatment of hair, including the strengthening of hair, prevention of hair loss, improved growth of hair, or the use of a formulation as described herein as a cosmetic hair treatment agent. It is especially advantageous for said use, if the additional component contain group B vitamins, particularly selected from the group comprising vitamins B2, B3, B5, B6, B7, and B12. In an advantageous embodiment, therefore, the present invention relates to a multi-component system as described herein in which the additional component contains group B vitamins, selected from the group comprising vitamins B2, B3, B5, B6, B7, and B12, particularly B7. It is especially advantageous for said use further, if the additional component contains minerals, particularly selected from the group comprising Mg, Zn, Cu, Fe, and I. In an advantageous embodiment, therefore, the present invention relates to a multi-component system as described herein in which the additional component contains minerals, selected from the group comprising Mg, Zn, Cu, Fe, and I.

The invention relates further to a formulation as described herein for the cosmetic (i.e., non-therapeutic) treatment of the skin, including the scalp, or the use of a formulation as described herein as a cosmetic skin treatment agent. It is especially advantageous for said use, if the additional component contains group E vitamins, particularly tocopherol. In an embodiment, therefore, the present invention relates to a multi-component system as described herein in which the additional component contains group E vitamins, preferably selected from the group comprising tocopherol.

The invention relates further to a formulation as described herein to suppress weight regain after a successful diet (yo-yo effect) and/or to prevent the restoration of fat depots or the use of a formulation as described herein to suppress the yo-yo effect and/or to prevent the restoration of fat depots. It is especially advantageous for said use, if the additional component contains amino acids, particularly selected from the group comprising arginine, lysine, and ornithine. In an advantageous embodiment, therefore, the present invention relates to a multi-component system as described herein in which the additional component contains amino acids, selected from the group comprising arginine, lysine, and ornithine.

The invention relates further to a formulation as described herein for the cosmetic (i.e., non-therapeutic) strengthening of muscles. Thus, the formulation described herein is suitable as a supplement in general training (athlete nutrition), as a supplement in muscle building training (bodybuilding), and/or as a supplementary food in extreme situations (mountain climbing, space flight). It is especially advantageous for this use, if the additional component contains all nutrient fractions essential for the human body in the correct proportion, particularly as described herein. In an advantageous embodiment, therefore, the present invention relates to a multi-component system as described herein in which the additional component contains nutrients, preferably selected from the group comprising, e.g., bases, proteins, vitamins, and carbohydrates. These nutrients are advantageously divided among a number of ZK.

Within the scope of the present invention, the formulation of the invention/food supplement of the invention/food of the invention are preferably taken according to a defined plan; accordingly, the formulation/food supplement/food can be adapted to this plan. Such a dosing schedule is characterized by (i) the duration of use (e.g., 12 months), time of intake (e.g., daily), and dosage (e.g., constant or increasing). The dosing schedule is adapted to the treatment goal and the person to be treated. The dosing schedule is established for the GK and each ZK independently of one another within the limits described herein. If more than one ZK is administered, the dosing schedule for the second and every other ZK is fully variable and independent of the dosing schedule for GK+ZK1.

Duration of use: According to the dosing schedule, the duration of use can vary over a broad range, preferably between 1.5 and 12 months. Although an even longer treatment is possible, it became apparent that no further improvement is achieved thereby. Likewise, a shorter treatment results in an incomplete effect. Advantageously, the treatment is designed either (i) as a long-term treatment, typically 9-15 months, or (ii) as an intensive treatment, typically 1.5-3 months, or (iii) as a standard treatment (which is especially advantageous for persons over 50 years of age), typically 4-8 months. The duration of use can also be shorter than the aforementioned 1.5 months; this is especially advantageous, if the formulations of the invention are given as co-therapy in conjunction with medications or natural medicines. Advantageously, the treatment is then designed (iv) as a co-therapy for medications/natural medicines, typically for a time period of 1 day to 2 months.

The duration of use, as described herein, relates to the main component (“basic interval”). The additional component(s) are also taken during the time period of the main component, but typically not before the start of the main component and not after the end of the main component. If a number of additional components are used, their durations of use can be established independently of one another, cf. FIG. 2.

Administration time: According to the dosing schedule, the administration time for GK and ZK can vary over a broad range, but repeats periodically. Thus, the administration can occur, for example, daily, 2× daily, 3× daily, weekly, or monthly. The administration time for GK and ZK can be established independently of one another, for example, GK 2× daily, ZK1 weekly, ZK2 daily.

In the protocol described herein, it is advantageous either (i) to take the GK 10 min-24 hours, preferably 30 min-12 hours, before the ZK or (ii) to take the GK 10 min-24 hours, preferably 30 min-12 hours, after the ZK. It is recommended to adhere to variant (i) or (ii) within the employed administration goal.

Dosage: According to the dosing schedule, the dosage can vary over a broad range. Basically, the dosage is adapted to the treatment goal and the person to be treated. The dosage for GK and ZK independently of one another can either be constant or variable. Typically, the dosage is such that the main component is administered in a greater amount than the additional component(s).

In one embodiment, the ratio GK:ZK>3:1, preferably >5:1. In a further embodiment, the ratio GK:ZK>7:1, preferably >10:1.

In an alternative embodiment, the ratio ZK:GK>3:1, preferably >5:1. In a further embodiment, the ratio ZK:GK>7:1, preferably >10:1.

The formulation described herein in the form of a multi-component system can be adapted to this dosage, for example, by already providing GK and ZK in the suitable proportion and/or by providing the ZK with inert components.

In protocol described herein, it is advantageous to keep the dosage of the GK constant during the basic interval.

In the protocol described herein, it is advantageous to increase the dosage of the ZK during the basic interval from a starting level to a maximum level and then again to reduce it to the starting level. The maximum level in this case can be greater than the starting level by the factor of 2-5.

In the protocol described herein, it is possible to take one or more (for example, 2) ZK during the treatment interval. If a number of ZK are taken, these can be taken simultaneously, sequentially, or alternately, cf. FIG. 3.

The invention therefore also relates to a formulation/food supplement/food, adapted for sequential administration, whereby
the main component is taken daily for a time period of 12 months and the additional component(s) daily from the 2^{nd}-11^{th} month, or
the main component is taken daily for a time period of 21 weeks and the additional component(s) daily from the 2^{nd}-20^{th} week, or
   the main component is taken daily for a time period of 4 months and the additional component(s) weekly for a time period of 4 months, or
   the main component is taken 2× daily for a time period of 10 weeks and the additional component(s) 1× daily from the 2^{nd} to 9^{th} week, or
   the main component is taken 2× daily for a time period of 13 weeks and the additional component(s) 1× daily from the 2^{nd} to 12^{th} week, or
   the main component is taken 1× daily for a time period of 13 weeks and the additional component(s) 2× daily from the 2^{nd} to 9^{th} week, or
   the main component is taken 1× daily for a time period of 7 weeks and the additional component 1× daily for a time period of 7 weeks and optionally a further additional component is taken once weekly for a time period of 7 weeks.

Such a sequential administration significantly improves the effectiveness, particularly the cosmetic effects described herein. The noted time periods can vary within a specific range; the person skilled in the art can easily determine this. Thus, it is possible to vary the GK by +/−20% and the ZK by +10% up to −90%.

It is especially advantageous, if the formulation/food supplement/food is adapted for sequential administration, in which the ZK is administered simultaneously or after, preferably after, the GK.

The invention in a third aspect relates to the use of the formulation of the invention as a medication.

It is assumed that about 10 medical conditions can be attributed to the deficiency of one of the aforementioned 90 nutrients. This means that about 900 diseases can be treated by suitable administration of these nutrients, particularly by the multi-component systems described herein. The invention therefore also relates to a formulation as described herein for use as a pharmaceutical product. Said aspect of the invention will be described in greater detail hereafter.

The terms “medication” or “pharmaceutical product” or “pharmaceutical composition” are known and comprise formulations that are administered to humans in order to achieve a therapeutic goal, particularly the healing, prevention, delaying, and/or alleviation of a pathological condition (particularly a disease as described herein).

The invention relates further to a formulation as described herein
for cleansing (i.e., elimination of toxins and/or harmful metabolic products from the human body);
for the treatment of acidosis (disturbance of the acid-base balance in the human body);
for the treatment of obesity;
for the treatment of diabetes;
for the treatment of mental illness (such as physical, emotional, mental exhaustion, burnout);
for the treatment of cancer (i.e., diseases in which body cells grow uncontrollably, as for the treatment of malignant tumors, for the treatment of malignant hemoblastosis);
for the treatment of cardiovascular diseases;
for the treatment of skin diseases (such as psoriasis, neurodermatitis; cellulitis, cellulite, acne);
to support wound healing;
for the treatment of joint diseases (including rheumatic complaints and gout);
for the treatment of dysfunctions and diseases of the liver;
for the treatment of dysfunctions and diseases of the kidneys;
for the treatment of autoimmune diseases;
for the treatment of HIV;
for the treatment of allergies or intolerances (such as lactose/fructose intolerance).

Without regarding oneself as bound to one theory, it is assumed that the aforementioned indications have a common feature, because they are caused or intensified by a deficiency in nutrients (especially minerals and vitamins). The medication of the invention can be adapted to specific indications and can therefore be developed in various directions. It has been found in various tests, as will be explained hereafter, that the medication of the invention is highly effective, particularly when the protocol described hereafter is used for administration.

The invention relates further to the use of a composition as described herein for the preparation of a medication for the treatment of a disease as described herein.

The invention relates further to a pharmaceutical composition/medication comprising a formulation as described herein.

The invention relates further to a treatment method for the treatment of humans, comprising the step of administering a therapeutically effective amount of a medication as described herein.

The formulation of the invention is suitable further to supplement and to improve existing therapies for the aforementioned diseases; the formulations are therefore suitable as a co-therapeutic agent or co-medication. In many cases, a synergistic effect is observed; i.e., the therapeutic success increases disproportionately to the corresponding monotherapies (having either only the administration of the conventional medication or only the administration of the formulation described herein). Thus, the invention also relates to a formulation as described herein as a co-therapeutic agent for the treatment of the aforementioned diseases. The administration of a known, conventional medication and the formulation described herein can occur simultaneously or sequentially.

Within the scope of the present invention, the formulation of the invention/the medication of the invention are advantageously administered in a defined dosing schedule (“protocol”); accordingly, the formulation/medication can be adapted to this protocol. The invention therefore also relates to a formulation/medication adapted for sequential use, whereby
the main component is administered daily for a time period of 12 months and the additional component(s) daily from the 2^{nd}-11^{th} month, or
the main component is administered daily for a time period of 21 weeks and the additional component(s) daily from the 2^{nd}-20^{th} week, or
the main component is administered daily for a time period of 4 months and the additional component(s) weekly for a time period of 4 months, or
the main component is administered 2× daily for a time period of 10 weeks and the additional component(s) 1× daily from the 2^{nd} to 9^{th} week, or
the main component is taken 2× daily for a time period of 13 weeks and the additional component(s) 1× daily from the 2^{nd} to 12^{th} week, or
the main component is taken 1× daily for a time period of 13 weeks and the additional component(s) 2× daily from the 2^{nd} to 9^{th} week, or
the main component is taken 1× daily for a time period of 7 weeks and the additional component 1× daily for a time period of 7 weeks and optionally a further additional component is taken once weekly for a time period of 7 weeks.

Such a sequential administration significantly improves the effectiveness, particularly the therapeutic effects described herein.

Such a protocol is characterized by (i) the duration of use (e.g., 12 months), time of intake (e.g., daily), and dosage (e.g., constant or increasing). The protocol is adapted to the treatment goal and the person to be treated. Further, the statements made in connection with the second aspect apply here accordingly.

Thus, in the protocol described herein, the GK and each ZK independently of one another can be established within the limits described herein.

Further, it is advantageous in the protocol described herein either (i) to take the GK 10 min-24 hours, preferably 30 min-12 hours, before the ZK or (ii) to take the GK 10 min-24 hours, preferably 30 min-12 hours, after the ZK. It is recommended to adhere to variant (i) or (ii) within a therapy.

Further, it is advantageous in the protocol described herein to keep the dosage of the GK constant during the treatment interval.

Further it is advantageous in the protocol described herein to increase the dosage of the ZK during the treatment interval from a starting level to a maximum level and then to reduce it again to the starting level. The maximum level in this case can be greater than the starting level by the factor of 2-5.

Further, it is advantageous in the protocol described herein to administer the GK as long as the pH in the body is above 7.5 and then to begin the dosing of the ZK. Tests have shown that the body pH should be raised significantly above 7.5 in test subjects in order to achieve the effects described herein. If the administration of the multi-component system was repeated directly after the end of dispensing, no satisfactory effect/s were achieved. Satisfactory results could be achieved again only if the test subject had passed through a general time interval and then was treated again according to the predefined protocol.

Further, it is possible in the protocol described herein to administer one or more (for example, 2) ZK during the treatment interval. If a number of ZK are taken, these can be administered simultaneously, sequentially, or alternately.

The examples cited hereafter are used to further illustrate the invention; they are not intended to limit the invention in any way, however.

### Example

### Dietetic Multiphase Food

The components GK1, ZK1, and ZK2 are used in combination as described hereafter. The components GK1, ZK1, and ZK2 can be administered in any formulation, for example, as a powder, tablet, gastro-resistant capsules/tablets, added to a slowly dissolving carrier, optionally gelatin, or a beverage, (e.g., suspension/syrup). In the case of minerals, the content is based on the corresponding element; these are used in the form of commercially available salts and/or can be worked up as described.

GK1 per 100 g of powder

| | | |
|---|---|---|
| Sodium carbonate/sodium bicarbonate | 31 | g |
| Calcium carbonate | 22.1 | g |
| Magnesium carbonate anhydrous | 17.6 | g |
| Siliceous earth, purified | 10.4 | g |
| Magnesium sulfate | 6.9 | g |
| Potassium/sodium tartrate | 4.7 | g |
| Sodium sulfate decahydrate | 1 | g |
| Manganese carbonate hydrate | 0.3 | g |
| L-Carnitine | 3 | g |
| Guarana or green tea extract powder | 2 | g |

ZK1 per tablet (effervescent tablets):

| Sodium hydrogen carbonate/bicarbonate | | 67 | mg |
|---|---|---|---|
| Carbohydrates: ca. | | 641 | mg |
| of these polyhydric alcohols | | 456 | mg |
| Vitamins: | Vitamin A | 400 | ug |
| | Vitamin D | 2.5 | ug |
| | Vitamin E | 10 | mg |
| | Vitamin C | 120 | mg |
| | Thiamine | 1.4 | mg |
| | Riboflavin | 1.6 | mg |
| | B1 | 2 | mg |
| | B2 | 2 | mg |
| | B6 | 2 | mg |
| | B12 | 1 | ug |
| | Niacin | 18 | mg |
| | Folic acid | 200 | ug |
| | Biotins | 150 | ug |
| | Pantothenic acid | 6 | mg |
| | Riboflavin | 0.6 | mg |
| Minerals: | Sodium | 115 | mg |
| | Calcium | 140 | mg |
| | Iron | 4.2 | mg |
| | Magnesium | 90 | mg |
| | Zinc | 4.5 | mg |
| | Manganese | 1.5 | mg |
| | Copper | 0.45 | mg |
| | Chromium | 30 | ug |
| | Molybdenum | 30 | ug |
| | Selenium | 15 | ug |
| | Chlorine | 77 | mg |
| | Iodine | 60 | ug |

ZK2 ingredients per 100 g:

| | | | |
|---|---|---|---|
| | Protein | 83 | g |
| | Carbohydrates | 2.4 | g |
| | Fat | 0.8 | g |
| | Lecithin | 5 | g |
| | Calcium orthophosphate | 24 | mg |
| Vitamins: | Vitamin B1 | 2 | mg |
| | Vitamin B2 | 2 | mg |
| | Vitamin B6 | 1.9 | mg |
| | Vitamin B12 | 0.005 | mg |
| | Vitamin C | 75 | mg |
| | Vitamin E | 12 | mg |
| | Pantothenate | 8 | mg |
| | Folic acid | 0.45 | mg |
| | Niacin | 15 | mg |
| Minerals: | Calcium | 1400 | mg |
| | E338 (P content given) | 700 | mg |
| | Zinc | 4 | mg |

Proteins and/or amino acids: e.g., L-glycine, L-alanine, L-valine, L-leucine, L-isoleucine, L-proline, L-phenylalanine, L-tyrosine, L-tryptophan, L-serine, L-threonine, L-cysteine, L-methionine, L-arginine, L-histidine, L-lysine, L-aspartic acid, L-glutamic acid, L-carnitine, creatinine, casein, and taurine.

The dietetic multiphase food is administered for weight reduction. In this test, the individuals were treated for the indicated time period and with the indicated compounds, as described hereafter.

| Comp. | 1^{st} month | 2^{nd} month | 3^{rd} month | | 4^{th} month | | 5^{th} month |
|---|---|---|---|---|---|---|---|
| GK1.1 | 1 ML | 2 ML | 3 | ML | 4 | ML | 3 ML |
| ZK1.1 | — | 2 Tab | 3 | Tab | 2 | Tab | — |
| ZK2.1 | — | — | | | — | | 2 ML |
| GK1.1 | 1 ML | 2 ML | ½ | ML | ½ | ML | — |

| | | | | | | |
|---|---|---|---|---|---|---|
| ZK1.1 | — | 2 Tab | 3 | Tab | — | — |

ML: 1 measuring spoon per day,
Tab: 1 tablet per day

The test subjects were examined after a defined period of time. The subjects were observed until improvement occurred. The vitality of the subjects was tested at specific time intervals.

The following results were achieved in this test; further, an overall finding of “good” was obtained in all cases.

| No./ | | Weight | | |
|---|---|---|---|---|
| gender | Age | beginning-end | Components | Duration |
| 1/F | 35 | 75-57 | GK, ZK1 | 3 months |
| 2/F | 25 | 67-60 | GK, ZK1 | 3 months |
| 3/M | 25 | 90-79 | GK, ZK1 | 4 months |
| 4/F | 55 | 70-60 | GK, ZK1 | 4 months |
| 5/F | 35 | 70-58 | GK, ZK1C | 3 months |
| 6/F | 36 | 76-60 | GK, ZK1, ZK2 | 3 months |
| 7/F | 56 | 69-61 | GK, ZK1, ZK2 | 4 months |
| 8/M | 30 | 78-67 | GK, ZK1, ZK2 | 5 months |
| 9/F | 33 | 67-56 | GK, ZK1, ZK2 | 5 months |
| 10/M | 28 | 78-70 | GK, ZK1 | 4 months |
| 11/M | 36 | 81-72 | GK, ZK1 | 3 months |
| 12/F | 39 | 73-62 | GK, ZK1 | 3 months |
| 12/M | 45 | 85-73 | GK, ZK1 | 3 months |

The results show that the dietetic food of the invention produces both a weight reduction and an improvement of the general state of health/vitality.

### Example

### Treatment of Malaise, Diseases

According to the invention, multi-component systems were tested further for cleansing and for the treatment of cellulitis, obesity, acidosis, rheumatism/gout, joint and muscle pain, hair loss, heart/circulatory/skin diseases, cancer, acne, kidney diseases, liver diseases, inflammations, alcohol intoxication (“hangover”), infectious diseases, environmentally induced diseases, age-related diseases, and for the treatment of mental disorders such as increasing the ability to concentrate, antidepressant effect, and burnout.

In these tests, the multi-component system of the invention was used either alone or as a supplement to a natural medicines (such as herbal extracts/omega-3, etc.) or as a supplement to medications (such as, e.g., antibiotics) to assess the mode of action.

It was found that the natural medicines or medications exhibited an improved effect (i.e., more rapid, more effective, and/or with fewer side effects) and thus an improved therapy was made possible.

It was found further that after the end of the test, weight was maintained for an average of 2 years and the state of health is evaluated as “excellent” by the test subjects. During this time, there was no additional physical activity or restrictions in terms of diet. Thus, a yo-yo effect is not observed.

### Example

### Dosing Schedules

In the dosing schedule, the details on the “Start” (S), “Duration” (D), and “end” (E) of the intended administration interval of the “GK” and “ZK” are variable within a range between 5-95%+/−, in conjunction with the indicated dosing protocol, preferably either 30%+/− and/or 50%+/− and/or 70%+/−. Depending on the type of utilization, the GK and ZK can be employed as described above.

As for ZK2 this is not associated with the specification of the interval for the GK/ZK1 and can be used from the first day onward.

The ZK can begin on the same day (as described herein) or on a day/week/month following thereupon, and can be repeated/used again after one or more interruptions.

### Example

### Effect of Sodium Carbonate/Sodium Hydrogen Bicarbonate

The special effect of Na2CO3 or NaHCO3 as a basic electrolyte is shown in the following examples A) to H). In these examples, results are presented which were observed in a comparison of the multi-component system of the invention (which contains Na2CO3 or NaHCO3) with an otherwise identical multi-component system that does not contain Na2CO3 or NaHCO3.

The unexpectedly marked effect of the aforementioned basic electrolyte is immediately apparent from these examples.

It is taken into account that sodium is of great importance in the regulation of the water balance in humans; there must be sufficient water in the “extracellular space” so that the circulation in humans functions.

In the tests, test subjects were tested periodically for about 7 years.

In the initial survey of the test subjects about their habits, it was found that they tried extensively to lose weight or to achieve a better general state of health but they did not get satisfactory results with any method (for example, with vegetables, fruit, fitness, fasting, sauna, drinking, or various ready-made products, for example, slimming products, etc.). Insofar as can be stated that in the tests until the use of the 2nd, etc., components ZK, except for about 10% which responded differently depending on personal characteristics, no decisive physical changes were identified. In general, a very good result was obtained and therefore the effectiveness of the product and the indicated management are assured.

A special effect on the body, which is to be called a washing machine effect, is clear from the performed tests:

A) It is clearly apparent that the use of sodium carbonate in combination with tolerable substances results in an alkaline saturation of the body, at a plasma pH of a maximum of 8.5.

B) It is clearly evident further that the use of vitamins in combination with tolerable substances activates a physical metabolic process which with each pass leads to an improvement of the recited conditions/indications, until the saturation of the body by acidic material, as a result of the release from the body by the use of sodium carbonate, and the addition of acidic substances from the outside, results in a plasma pH of about 6.5-7.5.

Treatment of Acidosis

The term acidosis generally describes a disturbance of the acid-base balance in the human body. The test series have clearly shown that during acidosis the body is greatly exhausted and overacidified. In order to restore the body rapidly and successfully, test series were performed with and without Na.

Test series 1: The subjects were treated with vitamins, trace elements, and/or natural medicines individually and in combination.

Result: No effect or more likely a negative effect, because in this state the body is incapable or capable only to a limited extent of taking up these acidic substances. Addition of general mineral substances and/or natural medicines also did not significantly accelerate healing or improvement.

Test series 2: The subjects were treated with sodium carbonate daily with at least 2 g, after which improvement occurred within a short time in about 1-2 weeks; alkaline saturation of the body, at a plasma pH of a maximum of 8.5.

Result: The uptake of mineral substances and natural medicines improved considerably and obviously. In a further step vitamins and trace elements could now be easily administered in very high doses as well.

100% successful.

Treatment of Obesity, Cellulitis, Cellulite, Acne

Test series 1: The subjects were treated with vitamins, trace elements (ZK), and/or natural medicines individually and in combination.

Result: No effect or more likely a negative effect, because in this state the body is incapable or capable only to a limited extent of taking up these acidic substances. Addition of general mineral substances and/or natural medicines also did not significantly accelerate healing or improvement.

Test series 2: The subjects were treated with sodium carbonate (GK) (at least 2 g daily), to which the tissue responded perceptibly within a short time, about 1-2 weeks. Alkaline saturation of the body, at plasma pH of a maximum of 8.5.

Result: The uptake of mineral substances and natural medicines improved considerably. This was also evident from the statements made by the subjects: Improvement in vitality occurred substantially in the 3^{rd}-6^{th} weeks, likewise an anti-aging effect.

In a further step vitamins and trace elements could now be easily administered in very high doses as well, the body responding very greatly alkalinically in this state in conjunction with acids, in terms of vitality and overall state.

It became apparent further that a treatment with high-dose vitamins and acids can be given in this way for a prolonged time. This was then also carried out for several weeks, whereby extensive weight reduction became apparent, without a yo-yo effect, over an observation period of about 2 years.

It became clearly apparent that addition of a number of basic electrolytes had a positive effect on the yo-yo effect, because otherwise in a specific intervention the body is greatly stressed and this is not recommended.

95% rehabilitation.

Treatment of Cancer

The term cancer describes diseases in which body cells grow uncontrollably, as for the treatment of malignant tumors, for the treatment of malignant hemoblastosis.

The subjects were treated under medical supervision with antibiotics and vitamins, trace elements, and/or natural medicines individually and in combination.

Result: No effect or more likely a negative effect, because in this state the body is incapable or capable only to a limited extent of taking up these acidic substances. Addition of general mineral substances and/or natural medicines also did not significantly accelerate healing or improvement.

The subjects were treated under medical supervision with sodium carbonate with at least 2 g daily, to which the tissue reacted perceptibly within a short time in about 1-2 weeks; alkaline saturation of the body, at a plasma pH of a maximum of 8.5.

In addition, the uptake of mineral substances and natural medicines (used internally and externally) improved considerably. This was evident from the statements made by the subjects: An improvement of the clinical picture was achieved, substantially an improvement in symptoms and swelling reduced by about 80% and an inflammatory reaction, which reveals the rejection or attack on tumor cells and to date achieved a reduction in tumor size by about 30%, and the effect of stable healing was achieved.

In another step vitamins and trace elements could now be easily administered in very high doses as well, the body responding alkalinically in this state in conjunction with acids, and the healing process being accelerated; in a first test it turned out that without the use of sodium carbonate there was no reaction or worsening, and also in terms of vitality and overall state.

It became apparent further that treatment with high-dose vitamins and acids can be given in this way for a prolonged time, for about 2-4 weeks, always alternating GK-ZK, which was then done and resulted in swelling with a subsequent subsidence of swelling; the tumor tissue separated from the normal healthy tissue and is clearly separated from other tissue as a freely movable element (like a kneecap), which can be repeated. In general, the variant described here is very advantageous in the removal of the tumor and for healing, because vitamins and trace elements are essential for recovery for the body.

This effect can be compared to a crank handle, which with each movement advances and engages by a tooth; in this case natural medicines, possibly bitter almond, hydrocyanic acid, optionally Taxaceae, and toxins, which achieved success in different test series, used internally and externally, have an effect on tumor tissue (Warthin's tumor), and a weakening and spasm-like reaction in the tumor which results in a slight subsidence of swelling and results in mild inflammation of the surrounding tissue, which is a matter of the dosage and what can be tolerated by a human, which is highly promising; thus by the dosing of two substances as mentioned, an optimal balance between the action of substance E (very stimulating) and, on the other hand, bitter almonds/hydrocyanic acid (very calming) can be dosed so that the general state is good (i.e., low in side effects) and both substances at the highest possible level that is overall possible in the body fundamentally serve the same purpose, comparable to engine tuning, to permit optimal toxin discharge which can flow easily through the body because of the inventive product, without physical damage or physical damage arising in the reduced state physical, which in a normal and ailing state is not possible and long-term damage of internal organs results; these are to be prevented by the multi-component system described herein, which represents an inventive principle. The freely accessible stabilization of the body can be used in a versatile manner, from survival kits to space food, from private use to clinical operations; said uses are therefore also the object of the invention.

Treatment of Cardiovascular Diseases

The subjects were treated with vitamins and trace elements and/or natural medicines individually and in combination.

Result: No effect or more likely a negative effect, because in this state the body is incapable or capable only to a limited extent of taking up these acidic substances. Addition of general mineral substances and/or natural medicines also did not significantly accelerate healing or improvement.

The subjects were treated with sodium carbonate with about at least 2 g daily, with improvement occurring within short time, within about 1-2 weeks. In addition, the uptake of mineral substances and natural medicines improved considerably.

In a further step vitamins and trace elements could now be easily administered in very high doses as well and the situation could be stabilized.

100% rehabilitation.

For the Treatment of Skin Diseases

The term skin diseases describes in particular psoriasis and neurodermatitis. Basically, in the case of psoriasis patients the inventive dosing schedule is very welcome, because it counterbalances the negative effects of very strong medications that are administered in such cases; these improve by 65-80% with the inventive dosing schedule.

The subjects were treated with vitamins and trace elements and/or natural medicines individually and in combination.

Result: No effect or more likely a negative effect, because in this state the body is incapable or capable only to a limited extent of taking up these acidic substances. Addition of general mineral substances and/or natural medicines also did not significantly accelerate healing or improvement.

The subjects were treated with sodium carbonate with about 2 g+daily, after which the normal effect is achieved as described above; 1-2 weeks.

In addition, the uptake of mineral substances and natural medicines was adapted to the characteristics of the psoriasis or neurodermatitis patients; these are administered combined in a GK and/or ZK optimally for the metabolic activity required by the patients.

Considerable improvement in about 80%,

Treatment of Dysfunctions and Diseases of the Liver

The subjects were treated with vitamins and trace elements.

Result: No effect or more likely a negative effect, because in this state the body is incapable or capable only to a limited extent of taking up these acidic substances. Addition of general mineral substances and/or natural medicines also did not significantly accelerate healing or improvement.

The subjects were treated alternately with sodium carbonate and amino acids after which improvement occurred within a short time of about 1-2 weeks.

A shortening of the healing time in liver disorders, for example, viral hepatitis, and in chronic epidemics was observed. Normalization of the acid-base balance in the liver by an increase in the alkaline property of the blood and the supplying of the tissue of the liver with oxygen and nutrients was achieved.

This was followed by the supplying of the body with about 40 important minerals and trace elements. They have inflammation-inhibiting and hepatoprotective properties because of the sulfur, magnesium, calcium, zinc, selenium, and other components.

Improvement in glucose and fat metabolism in liver cells, promotion of the storage of glycogen in the liver, and reduced accumulation of lipids and amino acids were achieved.

Bitter substances were then added. This results in an increase in the production and secretion of bile and in the pancreas in an improvement of digestion.

Promotion of the growth of beneficial intestinal flora and pre/probiotics for the elimination of parasites and yeasts was observed. In addition, the uptake of sodium carbonate, mineral substances, and natural medicines improved considerably.

Sodium sulfate is used for detoxification of the liver. This stimulates the function of the liver and gallbladder and thereby supports the elimination of toxins from the body.

The content of enzymes found in the liver is important. These are essential for metabolism in the liver.

In a further step vitamins and trace elements could now be easily administered in very high doses as well.

Treatment of Dysfunctions and Diseases of the Kidneys

The term disease of the kidneys describes in particular renal colic. The kidneys regulate the salt content in our body and apart from the intestines, lungs, and skin they are the most important excretory organs for metabolic waste products. They inhibit progression by treatment of the acidification with sodium carbonate.

The subjects were treated with sodium carbonate, vitamins, and trace elements.

Result: No effect or more likely a negative effect, because in this state the body is incapable or capable only to a limited extent of taking up these acidic substances. Addition of general mineral substances and/or natural medicines also did not significantly accelerate healing or improvement.

The subjects were treated in a balanced ratio with basic minerals, mineral substances, and natural medicines, about 2 g+daily, after which improvement occurs within a short time of about 1-2 weeks.

In addition, the uptake of sodium carbonate, vitamins, and trace elements improved considerably and these can support the healthy functioning of the kidneys and bladder.

In a further step sodium carbonate could be easily administered in very high doses as well and a normal function set in after rapid alleviation of the painful condition.

Support/Treatment of Other Indications

Other indications: Each of the following subjects can be assigned basically to one of the indicated approaches which yield results.

The following can be mentioned:

wound healing, pain therapy;

for the treatment of joint diseases (including rheumatic complaints and gout);

for the treatment of diabetes;

for the treatment of mental illness (such as physical, emotional, mental exhaustion, burnout);

for treatment during drug withdrawal;

for the treatment of autoimmune diseases,

for the treatment of HIV,

for the treatment of allergies or intolerances (such as lactose/fructose intolerance).

The invention being thus described, it will be obvious that the same may be varied in many ways. Such variations are not to be regarded as a departure from the spirit and scope of the invention, and all such modifications as would be obvious to one skilled in the art are to be included within the scope of the following claims.

What is claimed is:

1. A formulation in the form of a multi-component system containing a main component (GK) and one or more additional components (ZK) physically separated therefrom, wherein the main component contains basic electrolytes and optionally silicates, wherein the main component contains as the basic electrolyte at least 10% by weight sodium carbonate and/or sodium bicarbonate, and wherein the additional component contains vitamins and optionally trace elements, optionally amino acids/proteins, and optionally plant extracts.

2. The formulation according to claim 1, wherein the basic electrolytes are selected from the group comprising alkali salts and alkaline earth salts and/or the silicates are selected from the group comprising diatomaceous earth and/or the vitamins are selected from the group comprising vitamin A, group B vitamins, vitamins C, D, E, and K, and/or the trace elements are selected from the group comprising Ca-, Fe-, Mg-, Zn-, Mn-, Cr-, Mo-, Se-, Cl-, and I-containing compounds and/or the amino acids/proteins are selected from the group comprising proteinogenic amino acids, carnitine, creatinine, taurine and/or the plant extracts are selected from the group comprising guarana, caffeine, maca, and green tea extract.

3. The formulation according to claim 1, wherein the main component and the additional component(s) independently of one another are available as a solid dosage form, particularly powder, tablet, capsule and/or as a liquid dosage form, particularly syrup, suspension, or spray.

4. The formulation according to claim 1, wherein the main component contains more than 10% by weight of basic electrolyte and/or the additional component contains 0.01-1500% vitamins (based on the daily dose recommended by WHO).

5. The formulation according to claim 1 for use as a food supplement.

6. The formulation according to claim 5, adapted for sequential administration, wherein
the main component is taken daily for a time period of 12 months and the additional component(s) daily from the 2^{nd}-11^{th} month, or
the main component is taken daily for a time period of 21 weeks and the additional component(s) daily from the 2^{nd}-20^{th} week, or
the main component is taken daily for a time period of 4 months and the additional component(s) weekly for a time period of 4 months, or
the main component is taken 2× daily for a time period of 10 weeks and the additional component(s) 1× daily from the 2^{nd} to 9^{th} week, or
the main component is taken 2× daily for a time period of 13 weeks and the additional component(s) 1× daily from the 2^{nd} to 12^{th} week, or
the main component is taken 1× daily for a time period of 13 weeks and the additional component(s) 2× daily from the 2^{nd} to 9^{th} week, or
the main component is taken 1× daily for a time period of 7 weeks and the additional component 1× daily for a time period of 7 weeks and optionally a further additional component is taken once weekly for a time period of 7 weeks.

7. The formulation according to claim 1 for use as a pharmaceutical product.

8. The formulation according to claim 1 for the therapeutic treatment of obesity, cellulitis, and/or burnout.

9. The formulation according to claim 7 for use as a pharmaceutical product or according to claim 8 for the treatment of obesity, cellulitis, and/or burnout, adapted for sequential administration, wherein:
the main component is taken daily for a time period of 12 months and the additional component(s) daily from the 2^{nd}-11^{th} month, or
the main component is taken daily for a time period of 21 weeks and the additional component(s) daily from the 2^{nd}-20^{th} week, or
the main component is taken daily for a time period of 4 months and the additional component(s) weekly for a time period of 4 months, or
the main component is taken 2× daily for a time period of 10 weeks and the additional component(s) 1× daily from the 2^{nd}-9^{th} week, or
the main component is taken 2× daily for a time period of 13 weeks and the additional component(s) 1× daily from the 2^{nd} to 12^{th} week, or
the main component is taken 1× daily for a time period of 13 weeks and the additional component(s) 2× daily from the 2^{nd} to 9^{th} week, or
the main component is taken 1× daily for a time period of 7 weeks and the additional component 1× daily for a time period of 7 weeks and optionally a further additional component is taken once weekly for a time period of 7 weeks.

10. The formulation according to claim 1, wherein the additional component further comprises one or more medications.

11. A food supplement containing a formulation according to claim 1.

12. The food supplement according to claim 11, wherein GK and ZK independently of one another are present in the form of a powder, tablet, or syrup.

13. The food supplement according to claim 11, adapted for sequential administration, wherein:
the main component is taken daily for a time period of 12 months and the additional component(s) daily from the 2^{nd}-11^{th} month, or
the main component is taken daily for a time period of 21 weeks and the additional component(s) daily from the 2^{nd}-20^{th} week, or
the main component is taken daily for a time period of 4 months and the additional component(s) weekly for a time period of 4 months, or
the main component is taken 2× daily for a time period of 10 weeks and the additional component(s) 1× daily from the 2^{nd}-9^{th} week, or
the main component is taken 2× daily for a time period of 13 weeks and the additional component(s) 1× daily from the 2^{nd} to 12^{th} week, or
the main component is taken 1× daily for a time period of 13 weeks and the additional component(s) 2× daily from the 2^{nd} to 9^{th} week, or
the main component is taken 1× daily for a time period of 7 weeks and the additional component 1× daily for a time period of 7 weeks and optionally a further additional component is taken once weekly for a time period of 7 weeks.

14. Food, particularly in the form of a beverage and/or snack product, containing a formulation according to claim 1.

15. The food according to claim 14, adapted for sequential administration, wherein:
the main component is taken daily for a time period of 12 months and the additional component(s) daily from the 2^{nd}-11^{th} month, or
the main component is taken daily for a time period of 21 weeks and the additional component(s) daily from the 2^{nd}-20^{th} week, or
the main component is taken daily for a time period of 4 months and the additional component(s) weekly for a time period of 4 months, or
the main component is taken 2× daily for a time period of 10 weeks and the additional component(s) 1× daily from the 2^{nd}-9^{th} week, or
the main component is taken 2× daily for a time period of 13 weeks and the additional component(s) 1× daily from the 2^{nd} to 12^{th} week, or
the main component is taken 1× daily for a time period of 13 weeks and the additional component(s) 2× daily from the 2^{nd} to 9^{th} week, or
the main component is taken 1× daily for a time period of 7 weeks and the additional component 1× daily for a time period of 7 weeks and optionally a further additional component is taken once weekly for a time period of 7 weeks.
